(19) United States
(12) Patent Application Publication
(10) Pub. No.: US 2005/0089951 A1
(43) Pub. Date: Apr. 28, 2005

(54) Method for labeled amidation of biological molecule and method for analyzing biological molecule

(76) Inventors: Koichi Tanaka, Kyoto-shi (JP); Sadanori Sekiya, Kyoto-shi (JP); Yoshinao Wada, Osaka (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 10/969,900

(22) Filed: Oct. 22, 2004

(30) Foreign Application Priority Data
Oct. 24, 2003 (JP) 2003-365063

(51) Int. Cl. A61K 51/00; C12P 21/06

(52) U.S. Cl. 435/68.1; 530/409; 530/395; 424/1.11

(57) ABSTRACT

The present invention provides a method for improving the sensitivity of mass spectrometry for biological molecules. The present invention also provides a method for a rapid and simple analysis of biological samples by making use of the method for improving the sensitivity of mass spectrometry. A method for labeled amidation, comprising the step of reacting an amidating reagent labeled with ¹⁵N, an isotope of nitrogen, with the carboxyl group of a biological molecule. A method for analyzing a biological molecule, comprising the steps of performing amidation to a carboxyl group of a biological molecule to obtain an amidated form of the biological molecule, and subsequently subjecting the amidated form of the biological molecule to mass spectrometry. A labeled amidated form of a biological molecule wherein a carboxyl group of the biological molecule is amidated as ¹⁵N-labeled amide group.

### BACKGROUND OF THE INVENTION

1. Field of the Invention

The present invention relates to a basic research field in life science and, more specifically, to a technology for improving the sensitivity of mass spectrometry analysis of proteins, peptides, sugar chains, and other biological molecules.

2. Disclosure of the Related Art

Traditionally, MS/MS analysis using a tandem mass spectrometer has been employed in the structural analysis of proteins or peptides. For example, one technique described in Japanese Patent Laid-Open Publication No. Hei 10-90226 is designed to facilitate the generation of fragment ions of a protein or a peptide for mass spectrometry by chemically modifying the N-terminus or C-terminus of the protein or peptide with a charged amino acid.

The tandem mass spectrometer-based MS/MS analysis is also becoming a major technique in the structural analysis of sugar chains. In one technique, for example, elimination of sialic acid that occurs during the measurement of sialic acid-added sugar chains on a MALDI-TOF-MS apparatus is suppressed by esterifying the carboxyl group of the sialic acid with a methyl group (Rapid Communication in Mass Spectrometry. (10) 1996: 1027-32).

In the MS/MS analysis of proteins or peptides, a protein or a peptide is dominantly cleaved at the aspartic acid residues and glutamic acid residues, resulting in less fragment ions which is generated by cleavage at the other amino acid residues. This causes an entire decrease in the coverage of the fragment ions and makes the analysis difficult.

In the mass spectrometry of sialic acid-containing sugar chains using a MALDI ion source, sialic acid is likely to eliminate during the in-source decay (ISD) or post-source decay (PSD) of the ions, resulting in a decrease in the amounts of the molecular-related ions. Moreover, in the MS/MS analysis of sialic acid-containing sugar chains, sialic acid is dominantly eliminated, resulting in difficulty with generating the other fragment ions in amounts sufficient for analysis.

### SUMMARY OF THE INVENTION

Accordingly, it is an objective of the present invention to provide a method for improving the sensitivity of mass spectrometry for biological molecules. It is another objective of the present invention to provide a method for a rapid and simple analysis of biological samples by making use of the method for improving the sensitivity of mass spectrometry.

The present invention comprises the following aspects:

A first aspect of the present invention described in (1) through (10) below is directed to a method for labeled amidation of biological molecule.

(1) A method for labeled amidation, comprising the step of reacting an amidating reagent labeled with ¹⁵N, an isotope of nitrogen, with the carboxyl group of a biological molecule.

(2) The method according to the above (1), wherein the amidating reagent is [¹⁵N]ammonium chloride.

(3) A method for labeled amidation, comprising the step of reacting an amidating reagent labeled with ¹⁵N, an isotope of nitrogen, with the carboxyl group of a protein or a peptide.

(4) The method according to the above (3), wherein the carboxyl group is a carboxyl group of an acidic amino acid residue in the protein or a peptide.

(5) The method according to the above (4), wherein the acidic amino acid is selected from the group consisting of glutamic acid and aspartic acid.

(6) The method according to the above (3), wherein the amidating reagent is [¹⁵N]ammonium chloride.

(7) A method for labeled amidation, comprising the step of reacting an amidating reagent labeled with ¹⁵N, an isotope of nitrogen, with the carboxyl group of a sugar chain.

(8) The method according to the above (7), wherein the carboxyl group is a carboxyl group of an acidic sugar residue in the sugar chain.

(9) The method according to the above (8), wherein the acidic sugar is selected from the group consisting of sialic acid and muramic acid.

(10) The method according to the above (7), wherein the amidating reagent is [¹⁵N]ammonium chloride.

A second aspect of the present invention described in (11) through (16) below is directed to a method for analyzing a biological molecule.

(11) A method for analyzing a biological molecule, comprising the steps of performing amidation to a carboxyl group of a biological molecule to obtain an amidated form of the biological molecule, and subsequently subjecting the amidated form of the biological molecule to mass spectrometry.

(12) A method for analyzing a biological molecule, comprising the steps of:
performing amidation to a carboxyl group of a biological molecule by the method of labeled amidation according to the above (1), to obtain a labeled amidated form of the biological molecule; and subsequently
subjecting the labeled amidated form of the biological molecule to mass spectrometry.

(13) A method for analyzing a protein or a peptide, comprising the steps of performing amidation to a carboxyl group of a protein or a peptide to obtain an amidated form of the protein or a peptide, and subsequently subjecting the amidated form of the protein or a peptide to mass spectrometry.

(14) A method for analyzing a protein or a peptide, comprising the steps of:
performing amidation to a carboxyl group of a protein or a peptide by the method of labeled amidation according to the above (3) to obtain a labeled amidated form of the protein or a peptide; and subsequently
subjecting the labeled amidated form of the protein or a peptide to mass spectrometry.

(15) A method for analyzing a sugar chain, comprising the steps of performing amidation to a carboxyl group of a sugar chain to obtain an amidated form of the sugar chain, and subsequently subjecting the amidated form of the sugar chain to mass spectrometry.

(16) A method for analyzing a sugar chain, comprising the steps of:
performing amidation to a carboxyl group of a sugar chain by the method of labeled amidation according to the above (7), to obtain a labeled amidated form of the sugar chain; and subsequently
subjecting the labeled amidated form of the sugar chain to mass spectrometry.

A third aspect of the present invention described in (17) through (22) below is directed to an labeled amidated form of a biological molecule.

(17) A labeled amidated form of a biological molecule wherein a carboxyl group of the biological molecule is amidated as ¹⁵N-labeled amide group.

(18) The labeled amidated form according to the above (17), wherein the biological molecule is a biological molecule to be analyzed by mass spectrometry.

(19) A labeled amidated form of a protein or peptide wherein a carboxyl group of the protein or peptide is amidated as ¹⁵N-labeled amide group.

(20) The labeled amidated form according to the above (19), wherein the protein or peptide is a protein or peptide to be analyzed by mass spectrometry.

(21) A labeled amidated form of a sugar chain wherein a carboxyl group of the sugar chain is amidated as ¹⁵N-labeled amide group.

(22) The labeled amidated form according to the above (21), wherein the sugar chain is a sugar chain to be analyzed by mass spectrometry.

According to the present invention, there is provided a method for improving the sensitivity of mass spectrometry for biological molecules. There is also provided a method for a rapid and simple analysis of biological samples by making use of the method for improving the sensitivity of mass spectrometry.

### BRIEF DESCRIPTION OF THE DRAWINGS

FIG. 1 shows the results of mass spectrometry performed on a tryptic fragment of myoglobin which did not undergo amidation (VEADIAGHGQEVLIR, (m/z=1606)) (non-amidation), the same fragment which underwent unlabeled amidation (amidation (¹⁴N)), and the same fragment which underwent labeled amidation (amidation (¹⁵N)).

FIG. 2 shows respective MS spectra of SLNFP which did not undergo amidation (m/z=1144), SNLFP which underwent unlabeled amidation, and SNLFP which underwent labeled amidation.

FIG. 3 shows respective MS/MS spectra of SLNFP which did not undergo amidation (m/z=1144), SNLFP which underwent unlabeled amidation, and SNLFP which underwent labeled amidation.

### DETAILED DESCRIPTION OF THE INVENTION

The following description concerns the method for labeled amidation of a biological molecule.

The present invention is the method for converting a carboxyl group (—COOH) of a biological molecule into a ¹⁵N-labeled carbamoyl group (—CO¹⁵NH₂). Thus, the conversion of a carboxyl group into ¹⁵N-labeled carbamoyl group is written in the term “labeled amidation” in the present invention. Examples of the biological molecule include proteins, peptides, and sugar chains. The term “biological molecule” as used herein is intended to encompass any biological polymer.

First, as one example of the method for labeled amidation of the present invention, the method for labeled amidation of the protein or peptide is described. In this example, the protein or the peptide undergoes labeled amidation at the carboxyl groups of the acidic amino acid residues. The acidic amino acid may be any of α-amino acids, β-amino acids, γ-amino acids, or δ-amino acids. Examples of α-amino acids include aspartic acid and glutamic acid, which, according to the method of the present invention, are converted into asparagine and glutamine, respectively. In the present invention, when the proteins or peptides for labeled amidation include an unsubstituted C-terminus, the terminal carboxyl group also undergoes labeled amidation.

Any compound that can serve as an amidating reagent and is labeled with an isotope of nitrogen ¹⁵N may be used as the reagent for labeled amidation. For example, [¹⁵N]ammonium chloride and the like may be used. Also, any known amidation technique may be used in the present method for labeled amidation, provided that the labeled amidating reagent is used. For example, a protein or a peptide may be treated with a denaturing agent, when necessary, and then labeled amidation may be performed using the labeled amidating agent and a condensation agent. Examples of the condensation agent that may be used include various carbodiimides, such as 1-ethyl-3-(3-dimethylaminopropyl)carbodiimide (EDAC), N,N′-diisopropylcarbodiimide, cyanamide, N,N′-dicyclohexylcarbodiimide (DCC).

In the present invention, the carbodiimide process, which involves the use of a carbodiimide as the condensation agent, is preferably used. In this case, [¹⁵N]ammonium chloride is preferably used as the labeled amidating agent and 1-ethyl-3-(3-dimethylaminopropyl)carbodiimide (EDAC) is preferably used as the carbodiimide. In cases where the carbodiimide technique is employed, the labeled amidation is carried out, for example, in the following manner: A protein is dissolved in an aqueous solution of [¹⁵N]ammonium chloride in the presence of guanidine hydrochloride. To this solution, carbodiimide with guanidine hydrochloride added to it is added and the reaction is carried out. Preferably, 50 μl of approximately 500 mM to 1M [¹⁵N]ammonium chloride solution are used with respect to 600 pmol of the protein. The carbodiimide is preferably used in an amount of approximately 0.08 to 0.2 equivalents of the [¹⁵N]ammonium chloride. This reaction may be carried out at 4 to 30° C. over a time period of about 1 to 12 hours.

In this manner, the labeled amidation of protein or peptide may be carried out at its carboxyl groups. The method for labeled amidation of the present invention thus is particularly effective when a protein or a peptide of interest requires analysis by mass spectrometry.

Next, as another example of the method for labeled amidation of the present invention, the method for labeled amidation of a sugar chain is described. In this example, the sugar chain undergoes labeled amidation at the carboxyl groups of the acidic sugar residues. Examples of the acidic sugar include sialic acids and muramic acids. The method for labeled amidation of the present invention is particularly effective when used in conjunction with a sialic acid. Sialic acid is a general term for N-acylated neuraminic acids and their derivatives. Specific examples of sialic acid includes N-acetylneuraminic acid and N-glycolylneuraminic acid.

Any compound that can serve as an amidating reagent and is labeled with an isotope of nitrogen ¹⁵N may be used as the reagent for labeled amidation. For example, [¹⁵N]ammonium chloride and the like may be used. Also, any known amidation technique may be used in the present method for labeled amidation, provided that the labeled amidating reagent is used. For example, a sugar chain may undergoes labeled amidation using the labeled amidating agent and a condensation agent. Examples of the condensation agent that may be used include, aside from those described with reference to the labeled amidation of proteins or peptides, 4-(4,6-dimethoxy-1,3,5-triazine-2-yl)-4-methylmorpholinium chloride n-hydrate (DMT-MM). For example, in cases where [¹⁵N]ammonium chloride is used as the labeled amidating agent and DMT-MM is used as the condensation agent, [¹⁵N]ammonium chloride is used in an amount of approximately 50,000 to 500,000 equivalents of the sugar chain and DMT-MM is used in an amount of approximately 0.1 to 0.5 equivalents of the [¹⁵N]ammonium chloride. This reaction may be carried out at 30 to 60° C. over a time period of about 5 to 48 hours.

In this manner, the labeled amidation of sugar chain may be carried out. The method for labeled amidation of the present invention thus is particularly effective when a sugar chain of interest is analyzed by mass spectrometry.

The following description concerns the labeled amidated form of biological molecules.

The present invention is the amidated form of a biological molecule in which a carboxyl group of the biological is amidated as ¹⁵N-labeled amide group (i.e. ¹⁵N-labeled carbamoyl group). Thus, the situation to be amidated as ¹⁵N-labeled amide group is expressed in the term “labeled amidated” in the present invention. As described with reference to the method for labeled amidation, the amidated form of a biological molecule may be obtained by labeled amidation of the biological molecule. Examples of the biological molecules include proteins, peptides, and sugar chains. When a labeled amidated biological molecule is to be analyzed, the labeled amidated biological molecule is particularly useful for analysis by mass spectrometry.

The following description concerns the method for analyzing biological molecules in accordance with the present invention. The method for analyzing a biological molecule in accordance with the present invention is carried out by amidating the carboxyl groups (—COOH) of the biological molecule into carbamoyl groups (—CONH₂). Examples of the biological molecules to be analyzed by this method include proteins, peptides, and sugar chains.

The method of the present invention for analyzing biological molecules is now described with reference to one example in which the biological molecule to be analyzed is a protein or a peptide. In this example, the protein or peptide is amidated at the carboxyl groups of the acidic amino acid residues. The acidic amino acid may be any of α-amino acids, β-amino acids, γ-amino acids, or δ-amino acids. Examples of α-amino acids include aspartic acid and glutamic acid, which, according to the method of the present invention, are converted into asparagine and glutamine, respectively. In the present invention, when the protein or peptide to be amidated includes an unsubstituted C-terminus, the terminal carboxyl group is also amidated.

According to the method of the present invention for analyzing proteins or peptides, a protein or a peptide of interest is amidated at the carboxyl groups and the resulting amidated protein or amidated peptide is subjected to mass spectrometry analysis. Prior to mass spectrometry, the amidated protein or amidated peptide may be subjected to optional treatments, such as centrifugation, reduction followed by carboxymethylation, fragmentation, and desalting. These treatments can be performed by using known techniques.

In the analysis method of the present invention, the amidation is carried out by converting the carboxyl groups either into unlabeled carbamoyl groups (—CO¹⁴NH₂) or into labeled carbamoyl groups (—CO¹⁵NH₂). The conversion into unlabeled carbamoyl groups, i.e. unlabeled amidaiton may be performed by using any of the known amidation techniques, while the conversion into labeled carbamoyl groups, i.e. labeled amidation may be performed by using the above-described method for labeled amidation of a protein or a peptide.

One advantage that the amidation offers to the analysis method of the present invention is that acidic amino acid residues, such as aspartic acid residues and glutamic acid residues, that are susceptible to dominant cleavage, are no longer in existence by undergoing amidation, thereby increasing the likelihood that cleavage takes place at other amino acid residues. As a result, an increased number of fragment ions can be detected and more information for sequences can be obtained.

In the analysis method of the present invention, it is particularly preferred to carry out labeled amidation of proteins or peptides. The conversion of the —COOH groups into —CO¹⁵NH₂ groups by labeled amidation results in that the difference in mass between the protein or peptide of interest and the labeled amidated protein or peptide can be minimized to an extremely small value of 0.01301938 per one carboxylic group. In other words, the mass of the protein or the peptide before the labeled amidation is the same nominal mass as that after the labeled amidation. Thus, the mass data obtained by mass spectrometry of the labeled amidated protein or peptide are substantially reflecting the mass of the non-amidated protein or peptide. This facilitates analysis of the non-amidated form of the protein or the peptide based on these mass spectrometry data and thus makes it easy to make inquiries to the database for peptide mass fingerprint analysis (PMF) or MS/MS ion search analysis (MIS). As a result, the protein or peptide can be identified in a rapid and simple manner.

In comparison, when a protein or a peptide of interest undergoes unlabeled amidation, the difference in mass between the protein or peptide and the unlabeled amidated protein or peptide can be as large as about 1 per one carboxylic group. In some cases, the mass shift due to the unlabeled amidation can lead to an error in mass, which interferes with the PMF analysis or MIS analysis.

Another example of the method of the present invention for analyzing biological molecules is now described in which the biological molecule to be analyzed is a sugar chain. In this example, the sugar chain is amidated at the carboxyl groups of the acidic sugar residues. Examples of the acidic sugar include sialic acids and muramic acids. The resulting amidated sugar chains are then subjected to analysis by mass spectrometry.

In the analysis method of the present invention, the amidation is carried out by converting the carboxyl groups either into unlabeled carbamoyl groups (—CO¹⁴NH₂) or into labeled carbamoyl groups (—CO¹⁵NH₂). The conversion into unlabeled carbamoyl groups, i.e. unlabeled amidaiton may be performed by using any of the known amidation techniques, while the conversion into labeled carbamoyl groups, i.e. labeled amidation may be performed by using the above-described method for labeled amidation of a sugar chain.

One advantage that the amidation offers to the analysis method of the present invention is that when the sugar chain contains sialic acid, the sialic acid, that are susceptible to dominant cleavage, are no longer in existence by undergoing amidation. As a result, the elimination of the sialic acid that takes place during ISD or PSD is prevented and the amounts of ions associated with the molecular weight of the sugar chain are correspondingly increased, resulting in an increase in the sensitivity. In the MS/MS analysis, the amidation prevents the elimination of sialic acid, allowing other fragment ions to be detected. For this reason, more detailed structural analysis of a sugar chain can be conducted based on an MS/MS spectrum obtained by using sialic acid-containing ions as precursor ions and that obtained by using sialic acid-depleted ions as precursor ions.

In the analysis method of the present invention, it is particularly preferred to carry out labeled amidation of sugar chains. The conversion of the —COOH groups into —CO¹⁵NH₂ groups by labeled amidation results in that the difference in mass between the sugar chain of interest and the labeled amidated sugar chain can be minimized to an extremely small value of 0.01301938 per one carboxylic group. In other words, the mass of the sugar chain before the labeled amidation is the same nominal mass as that after the labeled amidation. Thus, the mass data obtained by mass spectrometry of the labeled amidated sugar chain are substantially reflecting the mass of the non-amidated sugar chain. This facilitates analysis of the non-amidated form of the sugar chain based on these mass spectrometry data. As a result, the sugar chain can be identified in a rapid and simple manner.

In comparison, when a sugar chain of interest undergoes unlabeled amidation, the difference in mass between the sugar chain and the unlabeled amidated sugar chain can be as large as about 1 per one carboxylic group. In some cases, the mass shift due to the unlabeled amidation can lead to an error in mass, which interferes with the database analysis.

### EXAMPLES

The present invention will now be described in further detail with reference to Examples, which are provided by way of example only and are not intended to limit the scope of the invention in any way.

### Example 1

Using mass spectrometry, a study was conducted to determine if myoglobin is amidated with [¹⁴N] ammonium chloride or [¹⁵N]ammonium chloride.

Un labeled amidation was carried out as follows: 600 pmol myoglobin was dissolved in 50 μl of 5M guanidine hydrochloride/1M ¹⁴NH₄Cl. To this solution, 15 μl of 5M guanidine hydrochloride/0.4M 1-ethyl-3-(3-dimethylaminopropyl)carbodiimide (EDAC) were added to make a reaction mixture. The reaction mixture was incubated at room temperature for 2 hours and was then transferred to Microcon YM-10 (Millipore) and 335 μl of 0.001M HCl added. This was followed by centrifugation at 14000 rpm at 4° C. for 70 min., addition of 40011 of 0.001M HCl, and additional centrifugation at 14000 rpm at 4° C. for 70min. Subsequently, 50 mM NH₄HCO₃ was added to a total volume of 100 μl, followed by addition of 1 μl of 1M DDT, a 1 hour incubation period at 56° C., addition of 5.5 μl of 1M iodoacetamide, and another 45 min. incubation period at room temperature. Following addition of 5 μl 100 mM CaCl₂, 6 pmol sequencing-grade modified trypsin (Promega) was added and the mixture was incubated at 37° C. for 16 hours. Subsequently, 10 μl 0.1 wt % TFA were added and the mixture was desalted/purified using ZipTip μ-C18 (Millipore) to give tryptic fragments of myoglobin.

Labeled amidation was carried out in the same manner as with the unlabeled amidation described above, to obtain digested fragments of myoglobin.

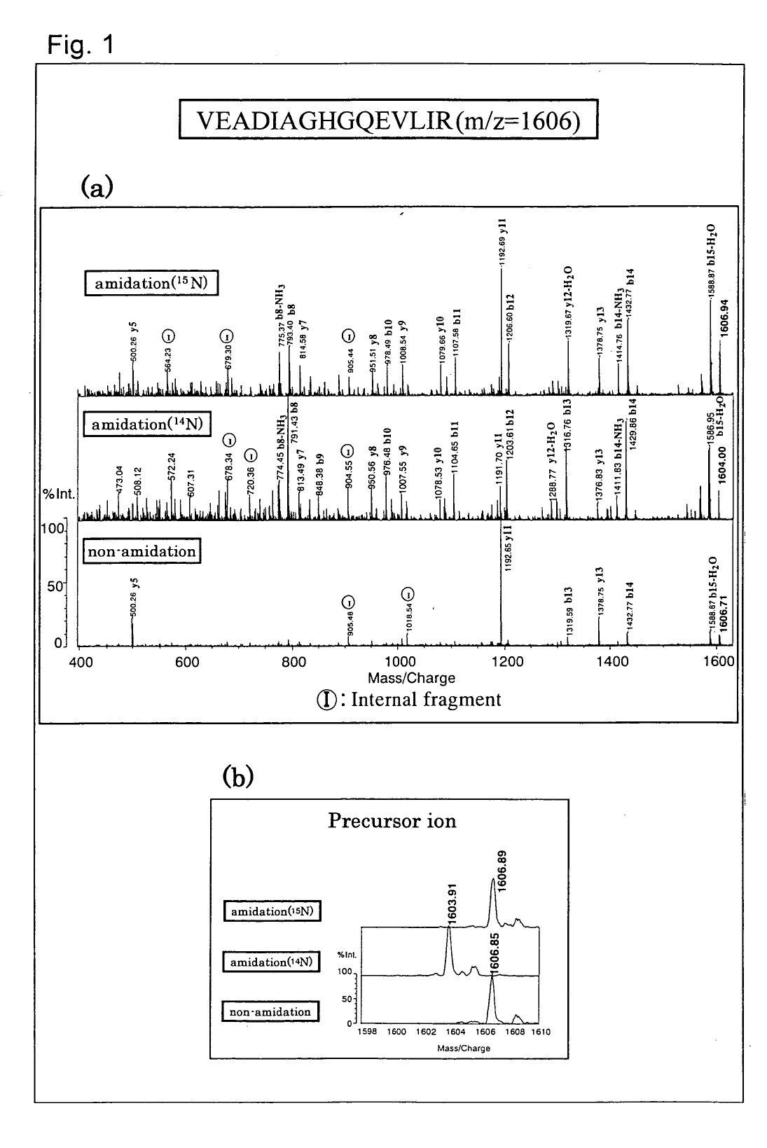

Using AXIMA-QIT (Shimadzu), a mass spectrometry analysis was performed on separately prepared non-amidated tryptic fragments of myoglobin, the tryptic fragments amidated with the unlabeled amidating agent, and the tryptic fragments amidated with the labeled amidating agent. Specifically, FIGS. 1(a) and (b) shows the results of mass spectrometry conducted on a particular tryptic fragment of myoglobin having a sequence of VEADIAGHGQEVLIR (SEQ ID NO: 1) (m/z=1606) (referred to simply as “peptide”, hereinafter), where respective spectra of the peptide which did not undergo amidation (non-amidation), the peptide which underwent unlabeled amidation (amidation (¹⁴N)), and the peptide which underwent labeled amidation (amidation (¹⁵N)) are compared with each other. In each spectrum, the horizontal axis corresponds to the mass-to-charge ratio (Mass/Charge) and the vertical axis corresponds to the ion intensity of fragment ions (% Int).

In FIG. 1, the MS/MS spectrum of the peptide which did not undergo amidation is shown at the bottom of (a) (non-amidation). This spectrum is obtained by using the precursor ions shown by the peak (m/z=1606.85) at the bottom of (b). The MS/MS spectrum of the peptide which underwent unlabeled amidation is shown in the middle of (a) (amidation (¹⁴N)). This spectrum is obtained by using the precursor ions shown by the peak (m/z=1603.91) in the middle of (b). Finally, the MS/MS spectrum of the peptide which underwent labeled amidation is shown at the top of (a) (amidation (¹⁵N)). This spectrum is obtained by using the precursor ions shown by the peak (m/z=1606.89) at the top of (b).

The mass number of the unlabeled amidated peptide is smaller than that of the non-amidated peptide by about 1 Da per one amidated carboxylic group. As shown by the MS/MS spectra in FIG. 1, when the mass number is compared between species of the fragment ions generated by the cleavage of the unlabeled amidated peptide and those generated by the cleavage of the non-amidated peptide at the same sites as in the cleavage of the unlabeled amidated peptide, the mass number of each fragment species is smaller in the unlabeled amidated peptide than in the non-amidated peptide by an amount corresponding to the number of the carboxylic groups which underwent unlabeled amidation. These observations indicate that the unlabeled amidation has successfully performed to the peptide.

Also, additional species of fragment ions other than those detected in the non-amidated peptides were detected, because the unlabeled amidation suppressed the dominant cleavage of the peptide at aspartic acid and glutamic acid residues. Addition to the above fragment ions, internal fragment ions were newly detected. Thus, the unlabeled amidation has been proven to increase the detection rate of the fragment ions.

The MS/MS spectrum of the peptide performed labeled amidation showed fragment ions that have substantially the same mass number as those detected in the MS/MS spectrum of the non-amidated peptide. Additional species of fragment ions other than those detected in the non-amidated peptides were detected. Addition to the above fragment ions, internal fragment ions were newly detected. These observations, together with the results obtained for the unlabeled amidation, indicate that the labeled amidation has successfully performed to the peptide. Thus, the labeled amidation has also been proven to suppress the dominant cleavage of the peptide at aspartic acid and glutamic acid residues and thereby increase the detection rate of the fragment ions.

Accordingly, it has been demonstrated that the amidation of the peptide increases the sensitivity of mass spectrometry analysis.

### Example 2

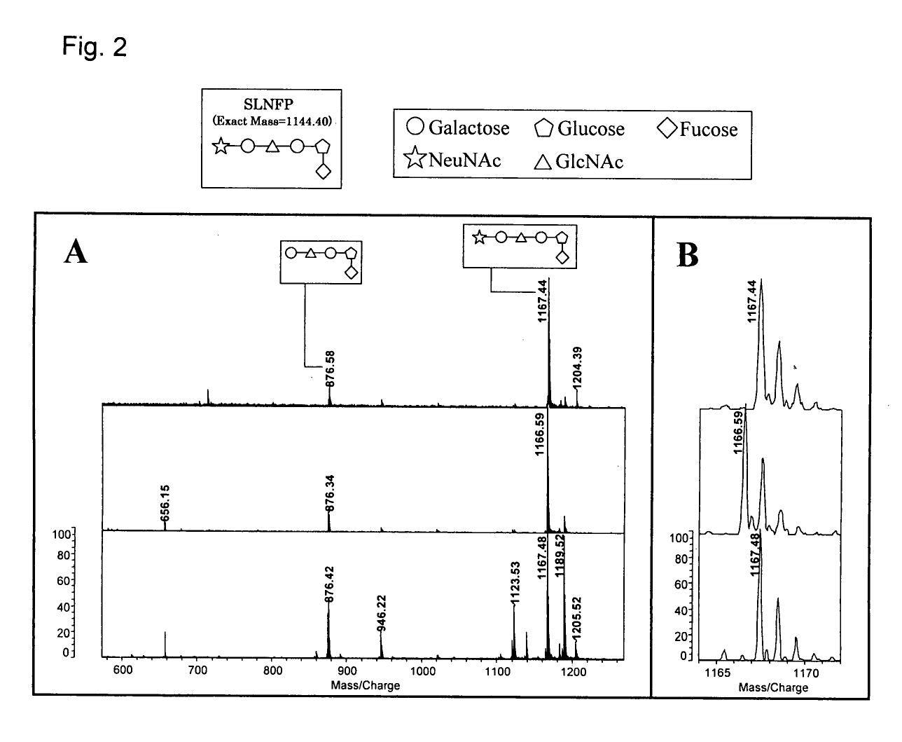

Using mass spectrometry, a study was conducted to determine if sialyllacto-N-fucopentaose (SLNFP, Exact Mass=1144.40), a sialic acid-containing sugar chain, is amidated with [¹⁴N]ammonium chloride or [¹⁵N]ammonium chloride. SLNFP is a sugar chain that consists of N-acetylneuramic acid (NeuNAc), galactose, N-acetylglucosamine (GlcNAc), glucose, and fucose. The structure of SLNFP is shown in FIG. 2.

The unlabeled amidation was carried out as follows: 1 μl of a 1 nmol/μl aqueous solution of sialyllacto-N-fucopentaose IV (SLNFP) was added to 50 μl of a 1M aqueous solution of ¹⁴NH₄Cl. To this mixture, 15 μl of a 1M aqueous solution of 4-(4,6-dimethoxy-1,3,5-triazine-2-yl)-4-methylmorpholinium chloride n-hydrate (DMT-MM) were added and the mixture (pH4.2) was incubated at 37° C. for 24 hours.

The reaction product was then subjected to gel filtration chromatography on a 5.5 cm high×1 cm diameter reaction vessel (Shimadzu) packed with 2.3 ml of Sephadex G-10 (Amersham bioscience) under atmospheric pressure. A 26th to 28th droplet (1 droplet is approximately 40 μl) was collected and was dried by centrifugation.

The dried product was dissolved in 5 μl H₂O. From this solution, a 1 μl aliquot was used for a mass spectrometry sample of SLNFP which underwent unlabeled amidation.

The labeled amidation was carried out in the same manner as described above except that ¹⁵NH₄Cl was used in place of ⁴NH₄Cl, to obtain a mass spectrometry sample of SLNFP which underwent labeled amidation.

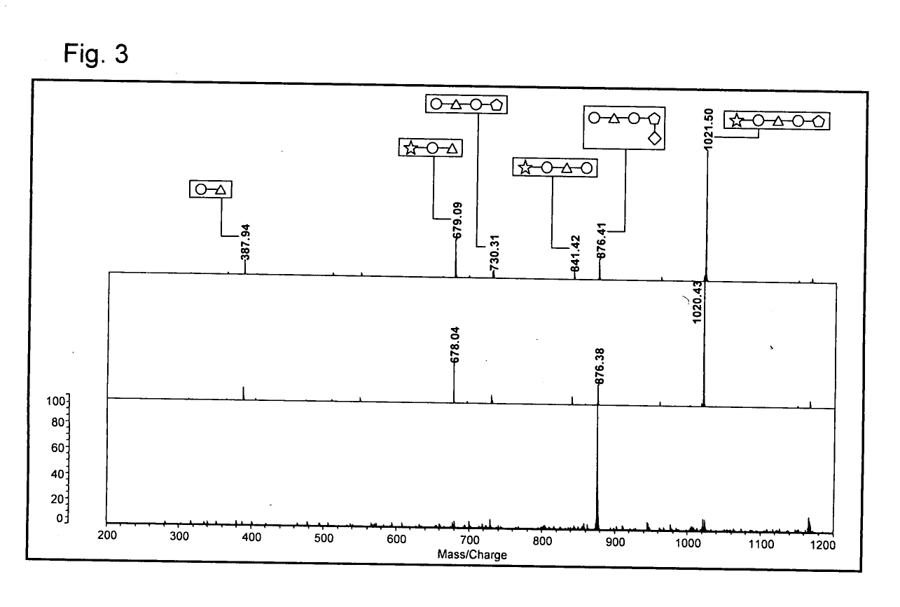

A mass spectrometry analysis was then performed on separately prepared SLNFP which did not undergo amidation, the SLNFP which underwent unlabeled amidation, and the SLNFP which underwent labeled amidation. The results are shown in FIGS. 2 and 3. FIG. 2 also shows the structure of SLNFP. A in FIG. 2 shows MS spectra obtained by AXIMA CFR (Shimadzu) while B in FIG. 2 shows enlarged spectra showing the peaks of the Na-adducted form of the molecular-related ions, [M+Na]⁺, shown in A. FIG. 3 shows MS/MS spectra obtained by AXIMA-QIT (Shimadzu) using [M+Na]⁺ as precursor ions. Of the spectra shown in FIGS. 2 and 3, the ones at the top are of the SLNFP amidated with the labeled amidating reagent, ¹⁵NH₄Cl, the ones in the middle are of the SLNFP amidated with the non-labeled amidating reagent, ¹⁴NH₄Cl, and the ones at the bottom are of the non-amidated SLNFP. In each spectrum, the horizontal axis corresponds to the mass-to-charge ratio (Mass/Charge) and the vertical axis corresponds to the ion intensity of fragment ions.

If a sugar chain undergoes unlabeled amidation, the mass number of the sugar chain which underwent unlabeled amidation will be smaller than that of the non-amidated sugar chain by about 1 Da per one carboxylic group converted by the unlabeled amidation. As can be seen from A and B in FIG. 2, the peak of the Na⁺-adducted molecular-related ion (m/z=1166.59) in the spectrum (middle) of the SLNFP amidated with the non-labeled amidating agent, ¹⁴NH₄Cl, has the smaller mass number than the peak of the Na⁺-adducted molecular-related ion (m/z=1167.48) in the spectrum (bottom) of the non-amidated SLNFP by 1 Da. This is equivalent to the carboxyl group of a single N-acetylneuramic acid in SLNFP converted to a carbamoyl group. This indicates that the unlabeled amidation with the unlabeled amidating agent, ¹⁴NH₄Cl, has successfully performed to the sugar chain.

As can further be seen from A and B in FIG. 2, the ion intensity of the peak of the ion generated by elimination of the sialic acid (m/z=876.34) shown in the spectrum (middle) of the SLNFP which underwent unlabeled amidation is lower than the ion intensity of the corresponding peak (m/z=876.42) shown in the spectrum (bottom) of the non-amidated SLNFP. This indicates that the elimination of sialic acid in ISD and PSD has significantly suppressed.

Referring now to FIG. 3, which shows the results of the MS/MS analysis, the ion intensity of peak of the ion generated by elimination of the sialic acid (m/z=near 875) shown in the spectrum (middle) of the SLNFP which underwent unlabeled amidation is lower than the ion intensity of the corresponding peak shown in the spectrum (bottom) of the non-amidated SLNFP. Also, the other fragment ions are newly detected in the spectrum (middle) of the SLNFP which underwent unlabeled amidation. These observations indicate that the unlabeled amidation has suppressed the elimination of the sialic acid from the sugar chain that takes place during mass spectrometry analysis, thereby increasing the coverage of the other fragment ions. This facilitates the determination of the sugar chain structure.

Referring to A in FIG. 2, the peak indicating the Na⁺-adducted molecular-related ions (m/z=1167.44) shown in the spectrum (top) of the SLNFP amidated with the labeled amidating agent, ¹⁵NH₄Cl, has substantially the same mass number as the corresponding peak (m/z=1167.48) shown in the spectrum (bottom) of the non-amidated SLNFP. Referring to FIG. 3, which shows the results of the MS/MS analysis, the ion intensity of the peak of the fragment ions generated by elimination of sialic acid shown in the spectrum (Top) of the SLNFP which underwent labeled amidation was lower than the ion intensity of the corresponding peak shown in the spectrum (bottom) of the non-amidated SLNFP, as was the ion intensity of the corresponding peak in the spectrum (middle) of the SLNFP which underwent unlabeled amidation. Also, the other fragment ions are newly detected in the spectrum of the SLNFP which underwent the labeled amidation. These observations indicate that the labeled amidation with labeled amidating agent, ⁵NH₄Cl, has successfully performed to the sugar chain. Further, it has been proven that the cleavage pattern of the sugar chain or the coverage of the ions is not affected by the presence of the isotope, except that the labeled amidation and the unlabeled amidation will result in the peaks with different masses due to the presence of the isotope.

Thus, the amidation of sugar chain has been proven to increase the sensitivity of the mass spectrometry analysis.

The above-described examples show concrete one mode within the scope of the present invention, however, the present invention can be carried out in various other modes. Therefore, the above-described examples are merely illustrative in all respects, and must not be construed as being restrictive. Further, the changes that fall within the equivalents of the claims are all within the scope of the present invention.

1. A method for labeled amidation, comprising the step of reacting an amidating reagent labeled with ¹⁵N, an isotope of nitrogen, with the carboxyl group of a biological molecule.

2. The method according to claim 1, wherein the amidating reagent is [¹⁵N]ammonium chloride.

3. A method for labeled amidation, comprising the step of reacting an amidating reagent labeled with ¹⁵N, an isotope of nitrogen, with the carboxyl group of a protein or a peptide.

4. The method according to claim 3, wherein the carboxyl group is a carboxyl group of an acidic amino acid residue in the protein or a peptide.

5. The method according to claim 4, wherein the acidic amino acid is selected from the group consisting of glutamic acid and aspartic acid.

6. The method according to claim 3, wherein the amidating reagent is [¹⁵N]ammonium chloride.

7. A method for labeled amidation, comprising the step of reacting an amidating reagent labeled with ¹⁵N, an isotope of nitrogen, with the carboxyl group of a sugar chain.

8. The method according to claim 7, wherein the carboxyl group is a carboxyl group of an acidic sugar residue in the sugar chain.

9. The method according to claim 8, wherein the acidic sugar is selected from the group consisting of sialic acid and muramic acid.

10. The method according to claim 7, wherein the amidating reagent is [¹⁵N]ammonium chloride.

11. A method for analyzing a biological molecule, comprising the steps of performing amidation to a carboxyl group of a biological molecule to obtain an amidated form of the biological molecule, and subsequently subjecting the amidated form of the biological molecule to mass spectrometry.

12. A method for analyzing a biological molecule, comprising the steps of:
performing amidation to a carboxyl group of a biological molecule by the method-of labeled amidation according to claim 1, to obtain a labeled amidated form of the biological molecule; and subsequently
subjecting the labeled amidated form of the biological molecule to mass spectrometry.

13. A method for analyzing a protein or a peptide, comprising the steps of performing amidation to a carboxyl group of a protein or a peptide to obtain an amidated form of the protein or a peptide, and subsequently subjecting the amidated form of the protein or a peptide to mass spectrometry.

14. A method for analyzing a protein or a peptide, comprising the steps of:
performing amidation to a carboxyl group of a protein or a peptide by the method of labeled amidation according to claim 3, to obtain a labeled amidated form of the protein or a peptide; and subsequently
subjecting the labeled amidated form of the protein or a peptide to mass spectrometry.

15. A method for analyzing a sugar chain, comprising the steps of performing amidation to a carboxyl group of a sugar chain to obtain an amidated form of the sugar chain, and subsequently subjecting the amidated form of the sugar chain to mass spectrometry.

16. A method for analyzing a sugar chain, comprising the steps of:
performing amidation to a carboxyl group of a sugar chain by the method of labeled amidation according to claim 7, to obtain a labeled amidated form of the sugar chain; and subsequently
subjecting the labeled amidated form of the sugar chain to mass spectrometry.

17. A labeled amidated form of a biological molecule wherein a carboxyl group of the biological molecule is amidated as ¹⁵N-labeled amide group.

18. The labeled amidated form according to claim 17, wherein the biological molecule is a biological molecule to be analyzed by mass spectrometry.

19. A labeled amidated form of a protein or peptide wherein a carboxyl group of the protein or peptide is amidated as ¹⁵N-labeled amide group.

20. The labeled amidated form according to claim 19, wherein the protein or peptide is a protein or peptide to be analyzed by mass spectrometry.

21. A labeled amidated form of a sugar chain wherein a carboxyl group of the sugar chain is amidated as ¹⁵N-labeled amide group.

22. The labeled amidated form according to claim 21, wherein the sugar chain is a sugar chain to be analyzed by mass spectrometry.


SEQUENCE LISTING (1 sequence)

SEQ ID NO: 1 - protein, 15 aa, Eguus caballus
VEADIAGHGQEVLIR